(19) United States
(12) Patent Application Publication
(10) Pub. No.: US 2010/0174133 A1
(43) Pub. Date: Jul. 8, 2010

(54) Apparatus and method for processing microscopic single cell biological specimens with a single microtool

(76) Inventors: Jacques Cohen, New York, NY (US); Tim Schimmel, Livingston, NJ (US); Michael D. Cecchi, Madison, CT (US)

Correspondence Address: William W. Jones, 6 Juniper Lane, Madison, CT, 06443, US

(21) Appl. No.: 12/592,630

(22) Filed: Nov. 30, 2009

Related U.S. Application Data
(60) Provisional application No. 61/200,462, filed on Dec. 1, 2008.

(51) Int. Cl. A61B 17/43; A61B 17/435

(52) U.S. Cl. 600/35

(57) ABSTRACT

An oocyte and embryo-positioning apparatus enables a plurality of oocytes, embryos or other cell specimens to be held and positioned for injection therein of fluid molecular reagents such as DNA, RNA, protein, cells, spermatozoa and cells; and removal of cells, fragments of cells, cytoplasm, nuclei and cellular organelles. The oocytes and embryos are kept separate from each other in open marked or unmarked well-compartments that are located in the culture container. Each well-compartment comprises one or more finger like structures that can position one oocyte or one embryo. The lanes between the digits of the fingers may be sloped allowing the oocytes or embryo to position at the narrowest and lowest point of the compartment. The method and oocytes and embryo-positioning apparatus of this invention includes a culturing container, such as a Petrie dish, in which the oocytes and embryos are held for micromanipulation. The Petrie dish preferably contains a plurality of the oocyte or embryo-holding finger like-structures which can be positioned in the Petrie dish in a predetermined pattern. For example, the well-compartments can be positioned in the Petrie dish as a strip of well-compartments between the 12 o'clock and 6 o'clock positions, or in any other planned deployment. Marked well-compartments can be identified by letters or numbers or symbols.

This application claims the benefit of U.S. Ser. No. 61/200,462, filed Dec. 1, 2008.

### TECHNICAL FIELD

This invention relates to a novel oocyte, embryo and specimen positioning apparatus and a method for inspection, observation, biopsy and the micromanipulation of oocytes, embryos and cells without the use of a second device, such as a holding pipette. The invention relates to the use of a series of structures within the apparatus that will allow the specimen to be placed into the apparatus and into the structures whereby the specimen is held in place may be easily located, and may be biopsied, manipulated or such without the use of a holding pipette. The apparatus allows the use of a single microtool, such as a micro injection pipette for the injection of materials, or a pipette to biopsy the specimen without a holding pipette to hold and stabilize the specimen during the procedure. The apparatus can be used for a wide range of specimens of animal and human cells, tissues, stem cells, embryos, oocytes and immature oocytes.

### BACKGROUND ART

Historically, culture dishes have been used for many procedures in mammalian embryology and cell culture. One procedure may be for the growth and culturing of specimens in defined environments involving incubation. Another procedure involves observation, manipulation and handling the gametes and embryos. These procedures may be of varying lengths of time, some are short and involve a specific activity such as the addition or removal of cryoprotectant for freezing and thawing, or micromanipulation such as intra-cytoplasmic sperm injection (ICSI) for improving fertilization in men with infertility, or to biopsy the specimen for preimplantation genetic diagnosis (PGD) in couples at risk for passing a genetic anomaly on to their offspring. Most current dishes are flat and are not designed to accommodate any particular procedure.

Prior art micromanipulation patents such as in Wakayama et al, U.S. Pat. No. 6,641,526, describe methods of using a holding pipette for micromanipulation. Wakayama et al specifically describes a method for freeze-drying spermatozoa to obtain at least one reconstituted spermatozoon whose head (nucleus) is capable of fertilizing an oocyte to produce a live offspring, after microsurgical injection into an oocyte. In this method they use one holding pipette and one injection microneedle.

Games et al, U.S. Pat. No. 6,717,031, specifically uses a holding pipette for microinjection of DNA into the male pronucleus of a zygote to create transgenic mice. Lindenberg, U.S. patent application number 20050239040A again shows the use of a holding pipette for ICSI to improve in vitro fertilization. The procedures described in the aforesaid patents all utilize a holding pipette for the procedures.

Current prior art of Petrie dishes and embryological culture dishes do not show any dishes, which are designed to eliminate the use of a holding pipette. The prior art shows dishes which are designed to require the presence of a holding pipette.

Stevens, U.S. Pat. No. 5,484,731 describes a multi-well in vitrofertilization plate for general use using a holding pipette.

It would be highly desirable to have an apparatus that would allow one to perform procedures on embryos, cells, oocytes, and similar specimens without the use of a holding pipette to hold the specimen in place.

It would also be desirable to have an apparatus with a defined structure or configuration that would allow one to perform procedures on embryos, cells, oocytes and similar specimens without the use of a holding pipette to hold the specimen in place.

It would be highly desirable to have a Petrie dish or specific dish with a configuration or structure that serves as a microscopic specimen holding device. This would allow the user of the dish to manipulate the microscopic specimen, be it an embryo, and oocyte, a cell, or the like, with only one hand and only one instrument, and without the need of a holding pipette.

### DISCLOSURE OF THE INVENTION

This invention relates to a culturing dish structure which enables a plurality of oocytes, cells and/or embryos to be held and positioned for injection or removal of a fluid without the need of a specimen holding pipette. The fluid can contain molecular reagents such as DNA, RNA, protein, cells and spermatozoa. The removed fluid can contain cells, fragments of cells, cytoplasm, nuclei and cellular organelles.

The individual oocytes, cells, or embryos (specimens) are kept separate from each other in open marked or unmarked well compartments that are located in the culturing dish. Each compartment comprises one or more finger like structures that can hold one oocyte, a clump of cells, or one embryo in a fixed position. The positioning of the specimens can be achieved using a single microneedle or microtool attached to a micromanipulation device. The configuration of the apparatus creates an area or lane having a flat bottom, and walls, whereby the lane has an opening and a narrowing between the walls.

The method and specimen-positioning apparatus of this invention may include a culturing container, such as a Petrie dish, in which the specimens may be captured for micromanipulation. The Petrie dish preferably contains a plurality of the specimen-holding finger-like structures which can be positioned in the Petrie dish in a predetermined pattern to form specimen capturing compartments in the Petrie dish. For example the capturing compartments can be positioned in the Petrie dish as a strip of compartments between the twelve o'clock and six o'clock positions, or in any other planned deployment in the dish. The compartment strip can be mirrored creating a duplicate row of finger-like projections in a mirror-like symmetry to be defined as mirrored structures which are composed of the same or varying components of the opposite side. The interdigitating narrowing areas allow for the positioning, holding, and micromanipulation of specimens, one in each compartment, without the need for a holding pipette to hold the specimens in place.

The method of this invention comprises the steps of placing the specimens in the container; moving each of the specimens into the lane structures of one of the interdigitating areas between the finger like projections; properly positioning each of the specimens with a microneedle connected to a micro manipulator; and using a suction device connected to this microneedle, when needed, for injection or biopsy. Conversely, the method of removing the specimens from the lane area will be comprised of the steps of: moving the microneedle adjacent to the specimen and pushing the specimen toward the narrowing end of the lane area; or using a standard pipette expelling culture fluid to direct the specimen toward the narrowing end of the lane. Cells or fragments removed can be placed in an alcove-like indentation in the structure for temporary storage to be used later or removed by a pipette. The reason for this alcove area is to place the biopsied cell or fragment away from the biopsied specimen so that both can be pipetted with a standard pipette separately allowing strict labeling and identification. The alcove is also the same focal length or the microscope in use, and in the same working plane as the micromanipulation device.

Additional variations of this invention can comprise marked capture compartments. These marked capture compartments can be identified by letters or numbers or symbols, for example, such as A, B, C, D, 1, 2, 3, 4, etc. inter alia. These markings can be etched or preprinted or simply written by the user.

The invention is structured in such a way that the dimensions of the area between the digits of the finger-like structures in the well can hold a mammalian oocyte, cell or embryo comfortably and securely so as to allow some compression and positioning of the specimen using a single micro manipulation micro pipette or micro needle tool. The compartments can be positioned in such a manner that oocytes and embryos will be safely separated allowing for stringent separation, manipulation, and administration. The individual oocytes, cell or embryos can be pipetted or otherwise moved into each compartment using any standard method and pipette tool which is normally used for embryological isolation and movement between wells and reagent micro droplets in a Petrie dish

The oocyte, cell or embryo can be removed from the restricting narrow areas of the compartment after the microprocedure by using a flow of fluid or culture medium released from a standard pipette or by gently probing the oocyte or embryo towards the tip of the narrowest end of the lane. A small alcove in each of the compartments may allow deposition and holding of biopsied cells or fragments from a biopsy micropipette after biopsy of the oocyte, cell or embryo. The biopsied cells or fragments can be pipetted safely using a narrow bored pipette. The dish may also contain separate wells for prepared samples of spermatozoa to be used for microinjection of one or more spermatozoa when the dish is used for intra-cytoplasmic sperm injection (ICSI). The dish may also contain other wells for washing oocytes or embryos before and after micromanipulation.

The apparatus of this invention may allow users to better and more effectively and efficiently perform the following procedures:

1) Intra-cytoplasmic sperm injection (ICSI) and other forms of assisted fertilization;
2) Polar body biopsy;
3) Assisted hatching, which is assisting the hatching process of a three day old embryo, or a blastocyst embryo, from the zona pellucida with a microtool, when the embryo is unable to hatch on its own;
4) Blastomere biopsy;
5) Blastocyst biopsy;
6) Manipulation of the zona pellucida (chemical, enzymatic or by laser);
7) Injecting solutions into the oocytes, blastomeres or blastocysts for transferring genes or other bio-molecules; and
8) Removing and injecting cytoplasm or organelles into oocytes or embryos.

These are some but not all procedures that the apparatus and method of this invention can be applied to.

These and other objects and advantages of the invention will become more readily apparent from the following detailed description of the invention, when taken in conjunction with the accompanying drawings in which:

### DESCRIPTION OF THE DRAWINGS

FIG. 1 is a plan view of a culturing dish formed in accordance with this invention showing a plurality of individual specimen positioning wells formed on the bottom of the dish;

FIG. 2 is a plan view similar to FIG. 1 but showing two rows of the positioning wells formed on the bottom of the dish;

FIG. 3 is a plan view of the dish but showing an embodiment thereof having back to back rows of contiguous specimen positioning structures formed on the bottom of the dish;

FIG. 4 is a plan view of a specimen positioning well in a culturing dish showing a version of a specimen positioning well which is particularly useful in biopsying oocytes, cell clumps or embryos, which well includes an alcove for the placement of the biopsied cell fragment or the like removed from the subject oocytes, cell clumps or embryos;

FIG. 5 is a fragmented plan view of one of the cell positioning structures, showing a specimen, in this case, an oocyte firmly positioned within the structure;

FIG. 6 is a fragmented plan view showing the commencement of the positioning procedure for one oocyte in a positioning structure;

FIG. 7 is a fragmented plan view similar to FIG. 6, but showing the oocyte as it is preliminarily rotated into a preferred position in the positioning structure;

FIG. 8 is a fragmented plan view similar to FIG. 7 but showing the oocyte firmly positioned in the positioning structure and an insemination needle approaching the oocyte;

FIG. 9 is a fragmented plan view similar to FIG. 8 but showing the needle piercing the oocyte to inseminate it with sperm; and

FIG. 10 is a fragmented plan view similar to FIGS. 7-9 but showing the inseminated oocyte after it has reached the embryo stage in the structure.

### DETAILED DESCRIPTION OF SPECIFIC EMBODIMENTS OF THE INVENTION

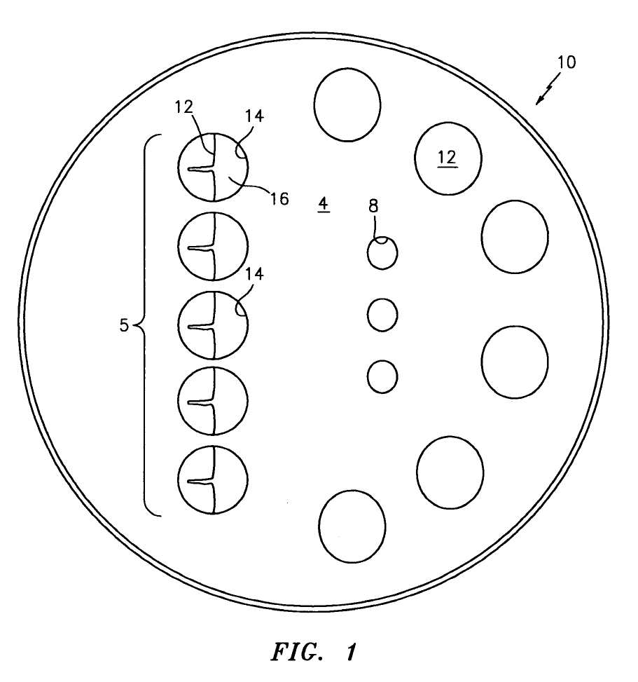

Referring now to FIG. 1 there is shown a plan view of the apparatus of this invention 12 inserted into a Petrie dish 10. The apparatus 12 is surrounded by walls 14 creating a small well 16. The walls 14 of the well 16 are taller than the apparatus 12. The walls 14 allow the user to place culture media and oils into the well area 16 to fully submerge the apparatus 12. There are five wells 16 in this embodiment of the invention. The number of wells 16 can be five, ten, or more, depending on relationship of the sizes of the wells 16 and the size of the dish 10, and the needs of the users will determine the exact number. Within the dish 10 are additional wells 8 which may be used to hold reagents, media, stem cells, embryos, sperm or other materials related to each procedure.

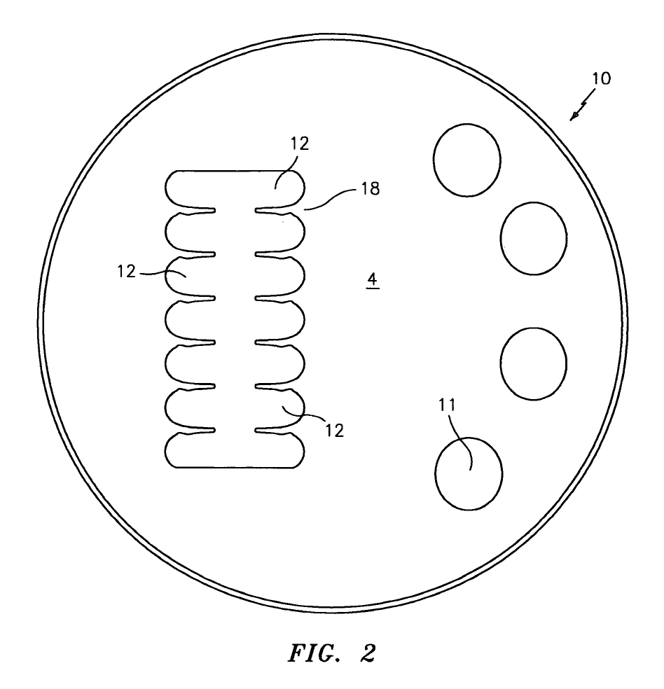

FIG. 2 shows a configuration of the apparatus 12 in a Petrie dish 10. In this example, the apparatus 12 consists of rows 18 of cell capture compartments, in a back to back fashion. This configuration illustrates the openings in the rows 18 of compartments in the apparatus.

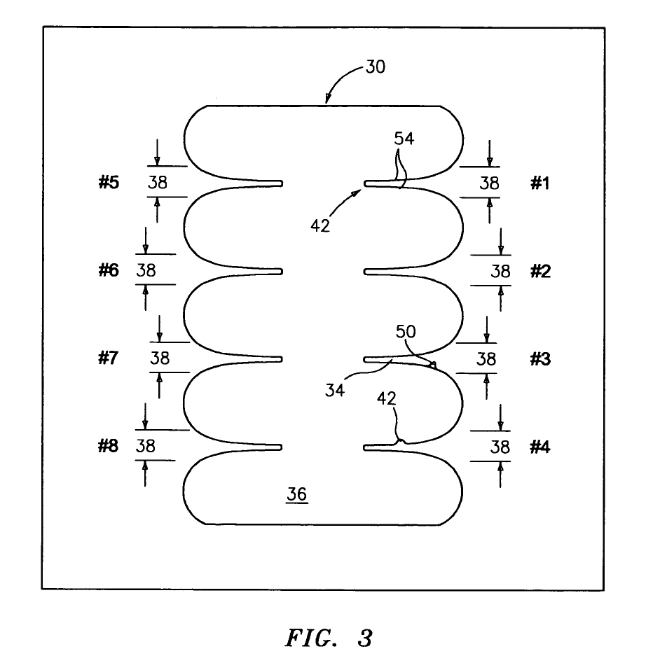

FIG. 3 is a plan view of an embodiment 30 of the apparatus 12. The embodiment 30 includes a raised portion 36 which rises above a lower surface 34. The lower portion 34 may be a part of the structure 30, or may be the base of the apparatus 12 which allows it to be placed in a Petrie dish (not shown). The raised portion 36 has slots or lanes 38 in which are created by walls 32. The lanes 38 have a bottom surface and an opening 40 which opens into a narrowed end 42. Within these lanes 38 there may be an alcove or recess 44 and a small dimple 50 in the walls 32, which alcove 44 and dimple 50 would allow the use to materials therein. The alcove 44 and dimple 50 allows the user to deposit materials in the apparatus 12 and still be in the focal region of a microscope (not shown) which will be used to monitor the boundaries of the lanes 38 and the micromanipulation of cell specimens by micro tools in the apparatus 12. The dimples 50 will enable the user to dislodge any cells or cellular material which might be stuck on the end of a microtool (not shown). The lanes 38 may be numbered 1, 2, 3 etc., or A, B, C etc. for identification and orientation.

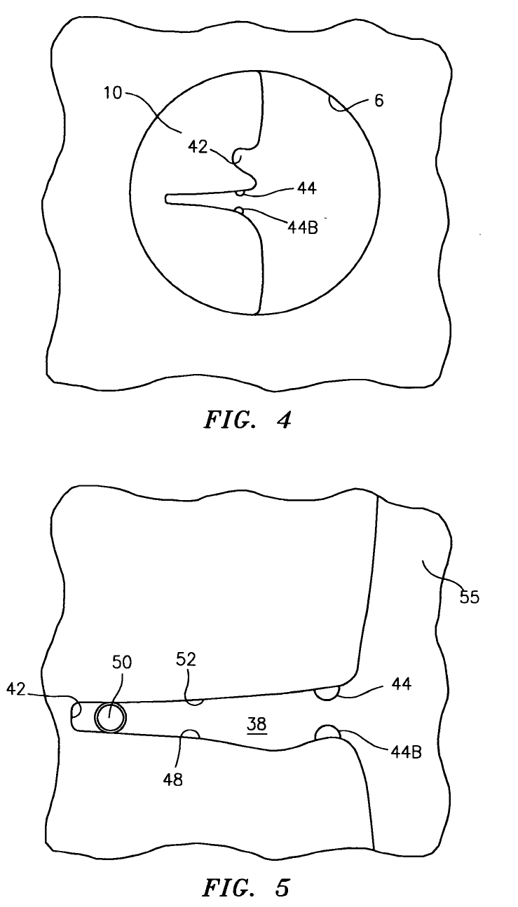

FIG. 4 shows a variation of the cell-capturing apparatus structure 12 which is useful for biopsying cells or embryos. The structure 12 includes walls 52, 52B and a bottom 34, creating a lane 58. The structure 12 includes an outer wall 14 which creates a well around the structure. The lane 58 is used for capturing and holding the cell or embryo after biopsy, as will be explained in greater detail hereinafter. The wall 44 may include a second smaller recess 42 which is operative to receive biopsied cell fragments from a cell being biopsied, or stem cells from an embryo that is being biopsied. The outer wall 14 allows culture media solutions to be contained in the created well.

FIG. 5 shows a more detailed top view of the apparatus 12. In this example there is a lane 56 which is the same level as the outer region 55. There are walls 52 in the raised portion 36 which create the lane 56. The lane 56 has an opening 38 and a narrowed end 42, and dimples 44 and 44B. The lane 56 includes a flat bottom 58 and walls 52 and 52B which are not parallel and which converge from the opening 38 to the narrow end 42. The opening 38 should be larger than the diameter of an oocyte, embryo or cell to be captured therein. The inner narrow end 42 will have a transverse dimension that is smaller than the diameter of the oocyte, embryo or cell to be captured therein. The narrowing effect will result in the specimen being squeezed in the vicinity of the narrow end 42 and thus safely held in place by the walls 52 of the lane 56. This will result in the specimen being snugly held in place between the walls 52 and 52B so that the user can perform the procedure in question with a single microtool.

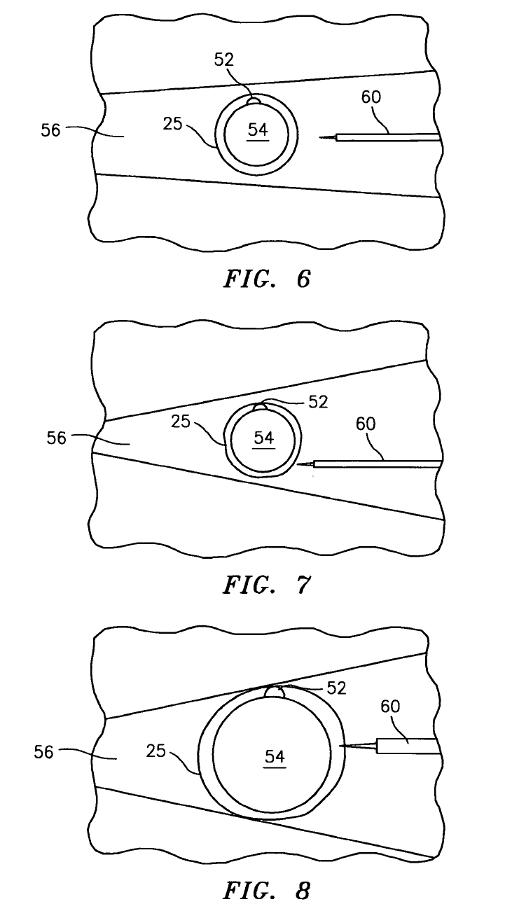

FIGS. 6-8 show one method for properly positioning an oocyte 54, or any other cell or embryo of appropriate size, in the lane 56. A microtool 60, such as a catheter or an aspiration needle, is used, and is aligned into the lane 56. In the specific example shown in FIGS. 6-8, the cell 54 is an oocyte. The microtool 60 can be used to direct the oocyte 54 into the lane 56. In this example, the microtool 60 is a micro injection needle. It will be noted that the mature oocyte 54 includes a polar body 28 and that the polar body 28 is at first randomly oriented in the lane 56. The microtool 60 can be used to properly orient the oocyte 54 so as to position the polar body 28, as shown in FIG. 7. In this orientation, the oocyte 54 can be inseminated by the micro injection needle 60 in a direction which is at a right angle to the position of the polar body 28 and not cause damage to the polar body 28.

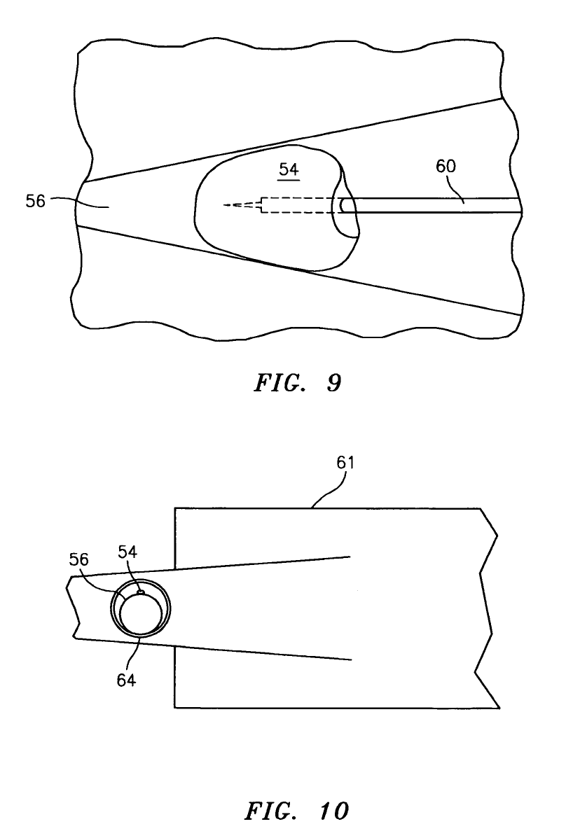

Once the oocyte 54 is properly oriented, keeping the polar body 28 oriented away from the micro injection needle 60, the insemination of the sperm is performed as shown in FIGS. 8 and 9. The micro injection needle 60 is inserted into the captured oocyte 54, as shown in FIG. 9 and the sperm is injected through the micro injection needle 60 into the oocyte 54 while the latter is held in place in the lane 56.

After insemination, the oocyte becomes an embryo as shown in FIG. 10 and blastomeres begin to develop inside of the embryo. This early embryonic development takes place within the zona pellucida 64 of the oocyte 54. The injected oocyte may then be picked up by the user with a pipette 61.

This dish structure allows each technician to use only one hand to perform a number of micromanipulation procedures on cells, embryos, and the like microscopic biological specimens, and does not require the use of a specimen holding straw with the other hand.

Including the zona pellucida, the average human oocyte is 150 microns in diameter, and falls with a range of 120-220 microns. The boundaries of the blastomeres in the embryo are well defined and their diameters range between 20 and 100 microns depending on their stage of cleavage. The early embryo has the same diameter as the oocyte because they are both contained within the zona pellucida.

Cells that can be biopsied are the polar bodies (by products of oocyte maturation) which range between 5 and 20 microns in diameter, and the blastomeres. Both oocytes and embryos may benefit from this procedure as the biopsied cell can be analyzed for genetic or metabolic content, for instance, chromosome number of genetic mutation as in preimplantation genetic diagnosis.

Techniques that can benefit from use with the dish structure of this invention include:

1) Intra-cytoplasmic sperm injection (ICSI) and other forms of assisted fertilization;
2) Polar body biopsy;
3) Assisted hatching, which is assisting the hatching process of a three day old embryo, or a blastocyst embryo, from the zona pellucida with a microtool when the embryo is unable to hatch on its own;
4) Blastomere biopsy;
5) Blastocyst biopsy;
6) Manipulation of the zona pellucida (chemical, enzymatic or by laser);
7) Injecting solutions into an oocyte, blastomere or blastocyst for transferring genes or other bio-molecules; and
8) Removing and injecting cytoplasm or organelles into oocytes or embryos.

While the invention has been described with respect to preferred embodiments, those skilled in the art will readily appreciate that various changes and/or modifications can be made to the invention without departing from the spirit or scope of the invention as defined by the appended claims.

What is claimed is:

1. An apparatus for micro manipulation of cell, oocyte or embryo specimens, said apparatus comprising: a container for positioning oocytes, cells or embryos, said container comprising one or more specimen-capturing compartments, wherein said specimen-capturing compartments comprise opposing walls forming one or more lanes, said walls forming a narrowing area in said lanes, wherein said narrowing area provides fixed positioning for micro manipulation of specimens without the need for a holding pipette during said micro manipulation operation.

2. The apparatus of claim 1 wherein said lanes have a sloping bottom wall with a lowest point at the narrowing area of said lanes.

3. The apparatus of claim 1 wherein said lanes have open tops so as to be accessible to a micromanipulation tool.

4. The apparatus of claim 1 wherein said specimen capturing compartments are disposed in wells in said container, said wells being operative to contain culturing media fluids during the micromanipulation of the specimens.

5. An assembly for use in micro manipulation of embryo, oocyte or other cell specimens, said assembly comprising:
a) a dish having at least one compartment for capturing and containing a single one of the specimens and for fixedly positioning said single specimen in a predetermined location in a lane in said dish; and
b) a micro tool for micro manipulation of said single specimen when the latter is in said predetermined location in said dish.

6. The assembly of claim 5 wherein said dish includes a well having an outer side wall forming a well in said dish.

7. The assembly of claim 6 wherein said lane has a downwardly tapered interior in which the cell settles by gravity, said predetermined location being formed by the lowest extremity of said tapered interior of said lane.

8. The assembly of claim 7 wherein said lane includes opposite converging side walls formed integrally with said outer side wall of said well, said side walls having opposed end surfaces which are spaced apart from each other to form an opening in said lane through which said micro tool can access a cell disposed in said lane.

9. The assembly of claim 8 wherein said dish includes a plurality of said lanes disposed on said outer side wall of said well.

10. The assembly of claim 8 wherein said micro tool is a tool for artificially inseminating oocytes disposed in said lanes.

11. An dish for use in micro manipulation of embryos, oocytes or other cell specimens, said dish comprising:
a) a well; and
b) a plurality of upwardly open lanes formed in said well, said lanes each having a bottom wall on which one cell settles by gravity into said lanes; said lanes each including opposed converging side walls, said side walls having opposed end surfaces which are spaced apart from each other to form openings in said lanes through which a micro tool can access a specimen disposed in each of said lanes; a narrowest end of each of said lanes being sized to conform to the size of a specific specimen whereby the specimen in question is confined in place in said narrowest end of each lane during micromanipulation of said specimen.

12. The dish of claim 11 wherein said well has a side wall which is taller than said side walls of said lanes whereby specimens in said lanes can be submerged by media solutions disposed in said well.

13. The dish of claim 11 wherein said bottom walls of said lanes tapers downwardly toward said narrowest end of said lanes.

14. The dish of claim 11 further comprising indentations adjacent to open ends of each of said lanes, said indentations forming alcoves for storage of specimen fragments or other materials for use in micromanipulation of specimens disposed in said lanes.

15. The dish of claim 11 further comprising projections in side walls of said lanes, said projections being operative to enable dislodgment of specimens from micro tools which are used in micromanipulation of specimens in said dish.

16. The dish of claim 11 further comprising auxiliary wells in said dish for use in storing media or specimens in said dish.

17. The dish of claim 11 wherein said lanes are distinguished one from another by different detectable symbols disposed in said dish adjacent to each of said lanes.
